(19) United States
(12) Patent Application Publication
(10) Pub. No.: US 2018/0353401 A1
(43) Pub. Date: Dec. 13, 2018

(54) A HAIR CARE COMPOSITION, PROCESS FOR PREPARING THE SAME AND METHOD OF USE THEREOF

(71) Applicant: ISP INVESTMENTS LLC, Wilmington, DE (US)

(72) Inventors: Solomon Tsigie WOSSENE, Riverdale, NJ (US); Michael A. TALLON, Aberdeen, NJ (US); Manuel GAMEZ-GARCIA, New City, NY (US); Alaa ALHARIZAH, Dover, NJ (US); David C. STREULI, Wayne, NJ (US); Linda C. FOLTIS, Nutley, NJ (US)

(73) Assignee: ISP INVESTMENTS LLC, Wilmington, DE (US)

(21) Appl. No.: 15/779,898

(22) PCT Filed: Dec. 1, 2016
(86) PCT No.: PCT/US16/64361

§ 371 Date: May 30, 2018

Related U.S. Application Data
(60) Provisional application No. 62/261,341, filed on Dec. 1, 2015.

(51) Int. Cl. A61K 8/35; A61K 8/34; A61K 8/27; A61K 8/26; A61K 8/43; A61K 8/46; A61K 8/81; A61K 8/73; A61K 8/04; A61Q 5/04; A61Q 5/06

(52) U.S. Cl. CPC A61K 8/35; A61K 2800/24; A61K 8/27; A61K 8/26; A61K 8/43; A61K 8/46; A61K 8/8176; A61K 8/8152; A61K 8/8158; A61K 8/731; A61K 8/732; A61K 8/042; A61K 8/046; A61Q 5/04; A61Q 5/06; A61K 8/345

(57) ABSTRACT

A hair straightening or hair styling composition comprising: (i) about 0.1 to about 70 wt. % of at least one hydroxy ketone, dihydroxy ketone or combinations thereof; (ii) about 0.1 to about 20 wt. % of at least one organic or inorganic base; (iii) optionally, about 0.1 to about 20 wt. % of at least one functional polymer; (iv) optionally, about 0.1 to about 20 wt. % of at least one diol, polyol, sugar, or combinations thereof; (v) optionally, about 0.1 to about 20 wt. % of at least one reducing agent; and (vi) about 40 to about 95 wt. % of at least one carrier; and wherein, the composition has a pH of about 5 to about 11. This application provides a process for preparing such hair styling or hair straightening composition and a method of use thereof.

### FIELD OF THE INVENTION

The present application relates to a hair care composition having a pH of about 5 to 11, and more particularly, to a hair straightening or hair styling composition comprising a hydroxy ketone, a base and carrier optionally in combination with a functional polymer, a diol, a polyol, a sugar, a reducing agent and/or an amino acid. The present application further relates to a method of preparing the hair straightening or styling composition.

### BACKGROUND OF THE INVENTION

Hair straightening or styling has become increasingly popular in view of hair styles which require relatively or perfectly straight hair. Hair protein, keratin forms a long helical chain like a very thin strand of rope. Groups of these thin strands form filaments. Groups of filaments, in turn, form the bulk of the hair strand's inner layer, called the cortex. Hair straighteners change how strands of keratin stick together. Different hair straightening methods target various chemical connections that hold keratin strands together.

Several types of hair straightening products currently used include formaldehyde, glyoxylic acid, dimethyl urea, α-hydroxy acids, their amides, α-keto acids, their amides, and glyoxylic amino acids such as carbo cysteine.

Brazilian Patent Application No. PI1101006 discloses the use of hydroxy acetone at a recommended pH range of 1.0 to 3.0 when applying to the hair. The highly acidic pH range is corrosive and degrades the hair fiber by hydrolysis of the native proteins in the hair. Individuals with oxidatively dyed hair cannot use this treatment as the highly acidic pH affects and reacts with the dyes and results in an obvious and unwanted color change in the dyed hair even before the heat activation step. Moreover, this extreme pH is also very irritating to the skin and scalp, and requires a professional salon to apply and prevent contact with the skin. This can result in excessive fees charged to the consumer in the salon to maintain a safe application/treatment onto the consumer.

U.S. Published Application No. 20130074862 assigned to L'Oréal S. A. discloses a process of straightening or relaxing hair by pre-alkalizing the hair before treating the hair with weak non-hydroxide base and oil. Weak inorganic bases include alkali metal phosphates and carbonates.

U.S. Published Application No. 20130139845 assigned to L'Oréal S. A. discloses a composition using at least one alpha-hydroxy and/or keto acid derivative. This composition does not contain any carboxylic acids/salts as straightening agents.

PCT Publication No. WO2014/016658A1 assigned to D Ottavi Adele et. al. discloses a composition requiring three components; dihydroxyacetic acid, hydroxy acetone and methylsulfonylmethane (dimethyl sulfone) to achieve the desired hair straightening effects. Methylsulfonylmethane (dimethyl sulfone) contains trace impurities of dimethylsulfide that are quite odoriferous and displeasing to the consumer. Therefore, a perfume is also needed to mask this unpleasant odor.

PCT Publication No. WO2012/084533A1 assigned to Henkel AG & Co. KGAA discloses a composition using dihydroxyacetone in a hydroalcoholic solution optionally containing a conditioning polymer and a fixative polymer for thermal straightening.

The various prior art techniques that are known in the prior art employ either low pH or high pH conditions, formaldehyde sprays are non-regulatory friendly, and/or require long processing times and unsafe procedures thus causing serious damage to the scalp and hair.

In view of the foregoing, clearly there remains a need for a safe and medium pH hair care composition which is capable of providing long lasting straightening effect within shorter processing time.

### SUMMARY OF THE INVENTION

The primary objective of the present application is to provide a hair straightening or hair styling composition which increases the degree of hair straightening or styling that lasts for at least 10 wash cycles and employs environment friendly ingredients that are compliant with global regulatory norms.

In one aspect, the present application provides a hair straightening or hair styling composition comprising: (i) about 0.1 to about 70 wt. % of at least one hydroxy ketone, dihydroxy ketone, or combinations thereof; (ii) about 0.1 to about 20 wt. % of at least one organic or at least one inorganic base; (iii) optionally, about 0.1 to about 20 wt. % of at least one functional polymer; (iv) optionally, about 0.1 to about 20 wt. % of at least one diol, polyol, sugar, or combinations thereof; (v) optionally, about 0.1 to about 20 wt. % of at least one reducing agent; and (vi) about 40 to about 95 wt. % of at least one carrier, wherein the composition has a pH of from about 5 to about 11.

According to another aspect of the present application, the above-described composition is further provided with (i) about 0.1 to about 20 wt. % of at least one protein or amino acid and (ii) about 0.1 to about 20 wt. % of at least one cosmetically acceptable ingredient selected from the group including but not limited to silicones, reducing agents, oxidizing agents, conditioning agents, humectants, pH adjusting buffers, petrolatum thickeners, mineral oils, emulsifiers, fragrances, and preservatives including phenoxyethanol, paraben, caprylyl glycol, iodopropynyl butyl carbamate, diazolidinyl urea and/or propylene glycol.

According to yet another aspect of the present application, the hydroxy ketone is selected from but not limited to α-hydroxyacetone, β-hydroxyacetone, 4-hydroxy-2-butanone, 1-hydroxy-2-butanone or combinations thereof.

According to yet another aspect of the present application, the organic or at least one inorganic base is an organic or an inorganic carbonates or bicarbonates, alkali metal hydroxides, sulfates, borates, manganates, aluminates, silicates, phosphates, carbonates alone or in combinations thereof.

According to another aspect of the present application, the diol is a C₃-C₁₂ diol selected from propanediol, butane diol, pentane diol, and hexane diol; polyol is C₃-C₁₂ polyol selected from the group consisting of glycerol, erythritol, threritol pentaerythritol, mannitol, maltitol, glucitolxylitol, sorbitol, diethylene glycol, triethylene glycol, and dipropylene glycol; and C₃-C₁₂ sugars are selected from the group consisting of glyceraldehyde, erythrose, threose, ribose, arabinose, xylose, lyxose, allolose, altrose, glucose, mannose, glucose, idose, galactose, talose, fructose, maltose, lactose, and the like.

According to yet another aspect of the present application, the above-described hair straightening or hair styling composition can be formulated into an emulsion, lotion, gel, vesicle dispersion, paste, cream, solid stick, mousse, shampoo, spray, balm, wipe, milk, foam, jellies, liquid, tonics, and/or enamel.

In another aspect, the present application provides a hair straightening or hair styling composition comprising: (i) about 0.1 to about 70 wt. % α-hydroxy acetone, β-hydroxyacetone, dihydroxy acetone, 4-hydroxy butanone, 1-hydroxy-2-butanone and combinations thereof; (ii) about 0.1 to about 20 wt. % sodium bicarbonate; (iii) optionally, about 0.1 to about 20 wt. % of at least one functional polymer; (iv) optionally, about 0.1 to about 20 wt. % of at least one diol, polyol, sugar, or combinations thereof; (v) optionally, about 0.1 to about 20 wt. % of at least one reducing agent; and (vi) about 40 to about 95 wt. % of at least one carrier; wherein said composition has a pH of about 5 to about 11.

In yet another aspect, the present application provides a method of straightening or styling hair, the method comprises: (a) washing the hair tresses with shampoo; (b) eliminating or reducing the moisture of hair tresses of step (a); (c) applying hair straightening or styling composition comprising: (i) about 0.1 to about 70 wt. % of at least one α-hydroxy ketone, β-hydroxy ketone, dihydroxy acetone, 4-hydroxy butanone, 1-hydroxy-2-butanone or combinations thereof; (ii) about 0.1 to about 20 wt. % of at least one organic or inorganic base; (iii) optionally, about 0.1 to about 20 wt. % of at least one functional polymer; (iv) optionally, about 0.1 to about 20 wt. % of at least one diol, polyol, sugar, or combinations thereof; (v) optionally, about 0.1 to about 20 wt. % of at least one reducing agent; and (vi) about 40 to about 95 wt. % of at least one carrier, wherein said composition has a pH of about 5 to about 11, to the dried or damp hair of step (b); (d) leaving the treated hair for about 15-30 minutes for penetration; (e) hot air drying the treated hair of step (d) by mechanically straightening with a slow uniform motion from root to tip; (f) ironing the hair resulting from step (e) for at least 4-8 times using flat iron pre-heated to 375-450° F.; and (g) allowing the hair tresses as of step (f) to remain as such for at least 15 to 20 minutes to obtain straightened or styled hair.

In another non-limiting aspect, the present application provides a hair styling or hair straightening composition that increases the degree or rate of hair straightening or styling that does not require highly acidic pH of about 1 to 4 or highly basic pH of about 12 to 14 conditions for straightening/styling and, requires less time to wash the straightened hair. The current process does not require a specific wait period of 24-36 hours before washing unlike the formaldehyde treatment or any other current hair straightening or styling techniques that are known.

### BRIEF DESCRIPTION OF THE FIGURES

Further embodiments of the present application can be understood with reference to the appended figures.

FIG. 1 illustrates effect of dihydroxyacetone alone or in combination with sodium bicarbonate on hair, wherein (A) is untreated hair; (B) is hair treated with dihydroxyacetone at pH 3.9; and (C) is hair treated with dihydroxyacetone and sodium bicarbonate at pH 7.5.

FIG. 2 illustrates effect of monohydroxyacetone alone or in combination with different bases on hair after one shampoo cycle, wherein (A) is untreated hair; (D) is hydroxy acetone at pH 3.5; (E) is hydroxy acetone and sodium bicarbonate at pH 8.1; (F) is hydroxy acetone and sodium tetra borate dehydrate at pH 8.3; (G) is hydroxy acetone and dibasic potassium phosphate at pH 8.2.

FIG. 3 illustrates effect of monohydroxyacetone and sodium bicarbonate on hair after 0, 1, 3, 5 and 10 wash cycles.

### DETAILED DESCRIPTION OF THE INVENTION

While this specification concludes with claims particularly pointing out and distinctly claiming that which is regarded as the invention, it is anticipated that the invention can be more readily understood through reading the following detailed description of the invention and study of the included examples.

The singular forms “a,” “an,” and “the” include plural forms unless the context clearly dictates otherwise specified or clearly implied to the contrary by the context in which the reference is made. The term “Comprising” and “Comprises of” includes the more restrictive claims such as “Consisting essentially of” and “Consisting of”.

All percentages, parts, proportions and ratios as used herein, are by weight of the total composition, unless otherwise specified. All such weights as they pertain to listed ingredients are based on the active level and, therefore; do not include solvents or by-products that may be included in commercially available materials, unless otherwise specified.

All references to singular characteristics or limitations of the present invention shall include the corresponding plural characteristic or limitation, and vice-versa, unless otherwise specified or clearly implied to the contrary by the context in which the reference is made.

As used herein, the term “cosmetically acceptable ingredient” means any ingredient/compound or mixture of ingredients/compounds or compositions that are typically employed to produce other aesthetic or cosmetic based desirable effects in personal care compositions. The preferred cosmetically acceptable excipients include but not limited to preservatives, antioxidants, chelating agents, sunscreen agents, proteins, amino acids, vitamins, dyes, hair coloring agents, plant extracts, humectants, fragrances, emollients, lubricants, butters, penetrants, thickeners, viscosity modifiers, polymers, resins, hair fixatives, film formers, surfactants, detergents, emulsifiers, opacifying agents, volatiles, propellants, liquid vehicles, carriers, salts, pH adjusting agents, neutralizing agents, buffers, hair conditioning agents, anti-static agents, anti-frizz agents, anti-dandruff agents, hair waving agents, hair straightening agents, relaxers, absorbents, fatty substances, gelling agents, moisturizers, hydrophilic or lipophilic active agent, fillers, reducing agents, cosmetic oils, perfumes, liquid vehicles, solvents, and combinations thereof.

The term “functional polymer” as described herein refers to IPUAC nomenclature i.e. a polymer that bears specified chemical groups or has specified physical, chemical, biological, pharmacological, or other uses which depends on specific chemical groups used for functionalization. Accordingly, the term “functional polymer” of the present application refers to a “polymer” which is of capable of demonstrating thickening, styling or conditioning properties.

Furthermore, the recitation of one or more preferred embodiments does not imply that other embodiments are not useful, and is not intended to exclude other embodiments from the scope of the invention.

The term “hair straightening” refers to the state where the natural curl of the hair has been removed and that the resulting straight configuration effectively resists reversion (i.e. resumption of a curly pattern) on being subjected to more than one washing, preferably at least four washings, and more preferably, at least eight washings or lasts until the scalp hair grows sufficiently to visibly warrant another hair straightening procedure.

The term “hair straightening iron” refers to device used in association with a combination of heat and pressure to straighten hair. It is generally composed of two flat heating elements, covered with various materials, between which a lock of hair is pressed at a time. Hair straightening irons are commercially available that are known to a person skilled in the relevant art. The source of energy of heating can be electric, solar, light, magnetic, chemical, and electromagnetic or combinations thereof.

The terms “include,” “includes,” and “including,” as used herein, are meant to be non-limiting and are understood to mean “comprise,” “comprises,” and “comprising,” respectively.

References herein to “one embodiment” or “one aspect” or “one version” or “one objective” of the application include one or more such embodiment, aspect, version or objective, unless the context clearly dictates otherwise.

All publications, articles, papers, patents, patent publications, and other references cited herein are hereby incorporated herein in their entirety for all purposes to the extent consistent with the disclosure herein.

The term “polymer” refers to a compound comprising repeating structural units (monomers) connected by covalent chemical bonds. Polymers may be further derivatized, crosslinked, grafted or end-capped. Non-limiting examples of polymers include copolymers, terpolymers, quaternary polymers, and homologues. The term “copolymer” refers to a polymer consisting essentially of two or more different types of repeating structural units (monomers).

The term “washed” and “washing” refers to rinsing of hair with water.

What is described herein is a hair straightening or hair styling composition comprising: (i) about 0.1 to about 70 wt. % of at least one α-hydroxy ketone, β-hydroxy ketone, dihydroxy acetone, 4-hydroxy butanone, 1-hydroxy-2-butanone or combinations thereof; (ii) about 0.1 to about 20 wt. % of at least one organic or at least one inorganic base; (iii) optionally, about 0.1 to about 20 wt. % of at least one functional polymer; and (iv) optionally, about 0.1 to about 20 wt. % of at least one diol, polyol, sugar, or combinations thereof; (v) optionally, about 0.1 to about 20 wt. % of at least one reducing agent; (iv) about 40 to about 95 wt. % of at least one carrier; wherein said composition has a pH of about 5 to about 11.

The present composition may further comprise at least one amino acid, and or at least one cosmetically acceptable personal care ingredient or additive.

A hydroxy ketone (often referred to simply as a ketol) is a functional group consisting of a ketone flanked by a hydroxyl group. In the two main classes, the hydroxyl group can be placed in the alpha position- an α-hydroxy ketone or in the beta position β-hydroxy ketone). Hydroxy ketones of present application having a general Formula 1 (general representation of both δ and β positions)

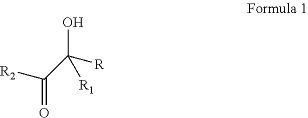

wherein R and R₁ are the same or different and are each H or a straight chain or branched and optionally substituted C₁ to C₁₂-alkyl radical, and R₂ is t-butyl, n-butyl, sec-butyl, n-propyl, isopropyl, an optionally heteroatom-substituted C₆-C₁₈ aryl, heteroaryl, C₆-C₁₈ arylalkyl, especially phenylmethyl, where phenyl can again be heteroatom substituted, or heteroalkyl or any combination thereof.

The suitable and non-limiting hydroxy ketone employed in the present application is present in an amount of from about 0.1 to about 70 wt. % of the total hair straightening or styling composition of the present application. Suitable hydroxyl ketones include α-hydroxyacetone, β-hydroxyacetone, 4-hydroxy-2-butanone, 1-hydroxy-2-butanone or combinations thereof.

Without being limited by theory, it is contemplated that the hydroxy ketone of the present application is capable of penetrating into the hair fiber's cortex to modify the hair fiber's natural shape while the styling polymer provides additional shape control, styling, and aesthetic qualities like body, shine, feel, conditioning and luster.

The suitable and non-limiting diols employed in the present application can be present in an amount of from about 0.1 to about 20 wt. % of the total hair straightening or styling composition. The diol can be a C₃-C₁₂ diol selected from propanediol, butane diol, pentane diol, and hexane diol.

The suitable and non-limiting polyol employed in the present application can be present in an amount of from about 0.1 to about 20 wt. % of the total hair straightening or styling composition. The polyol can be C₃-C₁₂ polyol selected from glycerol, erythritol, threritol pentaerythritol, mannitol, maltitol, glucitolxylitol, sorbitol, diethylene glycol, triethylene glycol, and dipropylene glycol.

The suitable and non-limiting sugars employed in the present application can be present in an amount of from about 0.1 to about 20 wt. % of the total hair straightening or styling composition. The sugar can have a carbon chain length of C₃-C₁₂ and can be selected from glyceraldehyde, erythrose, threose, ribose, arabinose, xylose, lyxose, allolose, altrose, glucose, mannose, glucose, idose, galactose, talose, fructose, maltose, lactose, and the like.

In another non-limiting embodiment, the hair straightening or styling composition of the present application comprises at least one organic or inorganic base in the concentration range of from about 0.1 to about 20 wt. % of the total composition. Suitable base for the hair care composition of the present application is an organic or inorganic carbonates or bicarbonates selected from the following non-limiting compounds including sodium bicarbonate, potassium bicarbonate, disodium or dipotassium carbonate, sodium carbonate, potassium carbonate; lithium carbonate, magnesium carbonate, calcium carbonate, zinc carbonate, aluminum carbonate, guanidine carbonate, imidazolium carbonate and imidazolium bicarbonate; diammonium carbonate, ammonium bicarbonate or ammonium sodium carbonate; alkyl or aryl ammonium bicarbonates selected from propylene carbonate; dialkyl ammonium carbonates or bicarbonates; alkylaryl ammonium carbonates or bicarbonates; diaryl ammonium bicarbonates, trialkyl ammonium carbonates or bicarbonates; alkylaryl ammonium carbonate or triaryl ammonium bicarbonates, hydroxyl alkyl or hydroxyl aryl ammonium bicarbonates; dihydroxyl alkyl ammonium carbonates or bicarbonates; hydroxyl alkyl hydroxyl aryl ammonium carbonates and bicarbonates; dihydroxyl hydroxyl aryl ammonium bicarbonates, trihydroxyl alkyl ammonium carbonates or bicarbonates; hydroxyl alkyl hydroxyl aryl ammonium carbonate; trihydroxyl aryl ammonium bicarbonates, where the alkyl or hydroxyl alkyl ammonium groups are linear or branched from carbon length C₁-C₃₀, hydrophobic chain lauryl, stearyl, behenyl and alkylaryl; or organic or inorganic alkali hydroxides, sulfates, borates, manganates, aluminates, silicates, phosphates, alone or in combination with the above predefined carbonates.

In another non-limiting embodiment, the hair straightening or styling composition of the present application comprises optionally, at least one functional polymer in the concentration range of from about 0.1 to about 20 wt. % of the total composition. The functional polymer as used herein is polymer which has a styling, thickening or conditioning properties. Additionally, wherein said functional polymer can possibly have either at least one individual property, for example, (i) styling and thickening, (ii) thickening and conditioning, (iii) styling and conditioning or (iv) styling, thickening and conditioning.

The functional polymers suitable for the purposes of the present application can include but are not limited to organic hair styling polymers that are well known in the relevant art. The organic styling polymers can be homopolymers, copolymers, terpolymers or other higher polymers, but should comprise one or more polymerizable hydrophobic monomers to render the resulting styling polymer hydrophobic and water-insoluble as defined herein. The styling polymers can possibly further comprise other water soluble, hydrophilic monomers provided that the resulting styling polymers have the requisite hydrophobicity and water insolubility.

In accordance with another embodiment of the present application, it is contemplated that an optional functional polymer is employed for preparing hair care composition of the present application, and wherein, a polymer or blend of polymers from synthetic and/or natural origin with weight average molecular weight from about 500 to about 20,000,000 daltons, containing at least one or more of the following monomeric components is selected the group including but not limited to styrenics, and/or sulfonated styrenics, acrylates, methacrylates, acrylamides, methacrylamides, vinyl lactams like vinyl pyrrolidone, vinyl caprolactam, vinyl amides, urethanes, ethoxylates, propoxylates, vinyl carboxylates, vinyl esters, vinyl ethers, maleates, itaconates, crotonates, and olefinics. Natural polymers are polysaccharides containing α-1,4 and/or β-1,4-glycosidic linkages with optionally α-1,6 and/or β-1,6 linkages, or optionally modified with (i) hydroxyalkyl, (ii) cationic functionality, (iii) hydrophobic groups including methyl, ethyl, propyl, and/or carboxymethyl groups, or (iv) hydrophilic groups including hydroxy ethyl, hydroxylpropyl, acetates, succinates, phosphates, sulfates and/or carbonates. Accordingly, in one specific embodiment, the natural polymer is cellulose, starch, guar, alginate, and/or carrageen gum.

In accordance with yet another embodiment of the present application, the functional polymer is cationic, anionic, amphoteric or nonionic in nature. Suitable functional polymers include but are not limited to acrylamide/sodium acrylate copolymer, sodium polyacrylate, acrylate/ammonium methacrylate copolymer, acrylic/acrylate copolymer, allyl stearate/vinyl acetate (VA) copolymer, aminoethylacrylate phosphate/acrylate copolymer, allyl stearate/VA copolymer, butyl ester of ethylene/maleic anhydride copolymer, isopropyl ester of methyl vinyl ether/maleic anhydride (PVM/MA) copolymer, PVM/MA decadiene crosspolymer, octyacrylamide/acrylate/butylaminoethylmethacrylate copolymer, octylacrylamide/acrylates copolymer, polyethylacrylate, poly(vinylpyrrolidone) (PVP), PVP/eicosene copolymer, PVP/ethyl methacrylate/methacrylic acid copolymer, Polyquaternium-37 PPG-1 Trideceth-6, glycol dicaprylate/dicaprate, PVP/hexadecene copolymer, PVP/VA copolymer, stearyl vinyl ether/maleic anhydride copolymer, vinyl acetate/crotonic acid copolymer, vinyl acetate/crotonic acid/vinyl neodecanoate copolymer, vinyl acetate/butyl maleate/Isobornyl acrylate copolymer, glycerin, glycerin acrylate/acrylic acid copolymer, guar, guar hydroxypropyltrimonium chloride or mixtures thereof.

Commercially available non-limiting examples of functional polymers include the following polymer products from Ashland Inc. (1) Cationic styling polymers with hair conditioning benefits—Styleze™ W Polymer, Styleze™ CC-10 (pseudo cationic), Gafquat™ 755 NP, and Gafquat™ 440; (2) Styling polymers with excellent high humidity curl retention-Styleze™ 2000, Allianz™ LT 120, Styleze™ W Polymer, and Advantage™ LCA; (3) Non-ionic styling polymers with broad ingredient compatibility, Polyvinylpyrrolidones such as PVP K-30, PVP K-60 and PVP K-90, vinylpyrrolidone/vinyl acetate copolymers such as PVP/VA (E, I or W) 735, PVP/VA (E or W) 635, PVP/VA (E or I) 535, PVP/VA (E or I) 335 and PVP/VA S-630, and poly (vinylpyrrolidone/dimethylaminoethylmethacrylate) polymers such as Copolymer 845/937.

The thickening polymers of the present application are capable of contributing to hair straightening; retaining the “hair style” as such for a longer duration, providing higher humidity control and increasing body or fullness.

In accordance with yet another embodiment, the present application further comprises from about 0.1 to about 20 wt. % of at least one reducing agents selected from, but not limited to, thioglycolate, thio sulfate, sulfite, bisulfite, sodium bisulfite and urea. These agents target disulfide bonds to create additional disulfide interchange and rearrangement.

In accordance with yet another embodiment, the present application further comprises from about 40 to about 95 wt. % of at least one carrier. The carrier is selected from the group consisting of water, alcohol, hydro alcoholic moiety, an emulsion, an ester, a hydrocarbon or an organic or inorganic oil carrier. An emulsion is a fluid colloidal system in which liquid droplets and/or liquid crystals are dispersed in a liquid. It is denoted by the symbol O/W (Oil in Water) if the continuous phase is an aqueous solution and by W/O (Water in Oil) if the continuous phase is an organic liquid (an ‘oil’). More complicated emulsions such as O/W/O (i.e. oil droplets contained within aqueous droplets dispersed in a continuous oil phase) are also possible.

In another non-limiting embodiment, the present application further comprises a cross-linking monomeric unit having alcohol, thiol, carboxylic acid or anhydride, amino or ammonium, modified silicone or siloxane, alkoxysilane, or sulfate group functionality.

In another non-limiting embodiment, the composition of the present application further comprises about 0.1 to about 20 wt. % of at least one protein dipeptide to decapeptide selected from following non-limiting group components including keratin amino acid, cysteine, histidine, lysine, arginine, aspartate, glutamate, asparagine, glutamine; alpha and/or beta-keratins, silk proteins, wheat protein, oat protein, milk protein, glycoprotein, soy protein, barley protein, quinoa protein, collagen protein, vegetable protein, chickpea protein, avacado protein, egg protein, honey protein, rice protein, casein, light protein, gelatin, or their hydrolyzed counterparts. Keratin Amino Acids are the Amino Acids that are extracted from the Keratin itself (amino acids make up a protein). They are then purified to make them stronger. Molecule size of the keratin amino acids allow it to penetrate the hair shaft to help repair damage (where as hydrolyzed keratin does not penetrate the hair). This is made from a mixture of amino acids, the most common in this mixture are Glutamic acid, Serine, Proline, Arginine, Threonine, and Aspartic acid.

Keratin Amino Acids is a powerful moisturizer that can penetrate the hair enhancing its conditioning effects. Since it has a composition similar to hair and is small enough to penetrate the hair shaft where it becomes highly substantive. It is ideal for hair that has suffered cuticle damage due to hair's increased porosity. It can help give the hair more sheen and increased volume.

In yet another embodiment, the present application comprises a hair straightening or styling composition which is formulated as per the requirement, and wherein, the non-limiting forms include emulsion, lotion, gel, vesicle dispersion, paste, cream, solid stick, mousse, shampoo, spray, balm, wipe, milk, foam, jellies, liquid, tonics, and/or enamel.

In yet another embodiment, the present application further comprises about 0.1 to about 20 wt. % of at least one cosmetically acceptable ingredient. Suitable personal care or cosmetically acceptable ingredients or conventional additives that are employed for preparing composition of the present application is well known in the relevant art and can be readily chosen by an artisan includes but not limited to functional silicones, acidifying or alkalizing pH adjusting agents and buffering agents; auxiliary fixatives and film formers that modify the on hair attributes of the polymer of the present application, such as gums, resins, polymers of synthetic or natural origin, and the like; auxiliary rheology modifiers such as viscosity enhancing polymeric thickeners or gellants; additives such as emulsifiers, detergents, emulsion stabilizers, waxes, dispersants, antioxidants, viscosity control agents such as solvents, electrolytes; hair and skin conditioning agents such as antistatic agents, synthetic oils, vegetable or animal oils, silicone oils, monomeric or polymeric quaternized ammonium salts, emollients, humectants, lubricants, sunscreen agents, and the like; chemical hair waving or straightening agents; hair colorants such as pigments and dyes for temporary, semi-permanent, or permanent hair dyeing; surfactants anionic, cationic, nonionic, amphoteric and zwitterionic surfactants; polymer film modifying agents plasticizers, humectants, tackifiers, detackifiers, wetting agents and the like, product finishing agents, chelating agents, sunscreen agents, proteins, amino acids, vitamins, opacifiers, pearlescing agents, resins, preservatives, fragrances, solubilizers, butter, penetrants, colorants pigments and dyes; UV absorbers, and the like; propellants (water miscible or water immiscible) such as fluorinated hydrocarbons, liquid volatile hydrocarbons, compressed gases, other additives such as volatiles, propellants, liquid vehicles, carriers, salts, anti-static agents, anti-frizz agents, anti-dandruff agents, hair waving agents, relaxers, absorbents, fatty substances, moisturizers, hydrophilic or lipophilic active agent, reducing agents, and combinations thereof.

The suitable non-limiting moisturizers employed in the present application include, but are not limited to, glycols, glycerols, propylene glycol, diethylene glycol monoethyl ether, sorbitol, sodium salt of pyroglutamic acid, glycerol, glycerol derivatives, dimethicone polyol, glycerin, trehalose, sorbitol, maltitol, dipropylene glycol, 1,3-butylene glycol, sodium hyaluronate, and the like.

The functional silicones can be selected from the group consisting of silicone quat, silicone fluid and silicone wax. These silicones are able to decrease friction during hair combing and flat ironing, smooth gliding; increase conditioning and smoothing to hair by adding flexibility; help reducing heat damage and style retention; contributes to shine depending on the refractive index.

The oxidizing agents can be selected from, but are not limited to, hydrogen peroxide, bromides and per acetic acid to fix the disulfide bond back.

The conditioning agents can be selected from, but are not limited to, polymeric and small quaternary ammonium compounds such as tertiary amines quaternium and polyquaternium compounds.

The humectants can be selected from, but are not limited to glycols, sugars, oils, silicones, emollients and proteins. These humectants create a cage effect by adding moisture to the hair.

The pH adjusting buffers can be selected from, but are not limited to, glyoxylic acid, maleic acids, succinic acid, formic acid and other organic and inorganic acids. These buffers promote active penetration of the composition of the present application and facilitate the reactions if any for effective results of the composition.

The preservatives can be selected from, but are not limited to, propylene glycol, paraben, methyl paraben, phenoxyethanol, caprylyl glycol, iodopropynyl butyl carbamate, and diazolidinyl urea.

According to one another embodiment of the present application, a catalyst is employed to facilitate the reaction speed of active ingredients, and wherein, the suitable catalyst can be non-metallic (N-methylimidazole, Perfluoroalkanesulfonates, N-dimethyl amino pyridine) or metallic (tin, aluminum, iron, scandium, yittrium, zinc, bismuth, ruthenium, zirconocene) in nature.

Further, the present composition also may comprise fatty alcohols or waxes as structuring agents, anionic or non-anionic surfactants or wetting agents, petrolatum as thickeners protectants, mineral oils as lubricating agents, mica or silicas, bentonite, or other inorganic powders as thickening agents and heat transfer agents.

In another non-limiting embodiment, the present application discloses a hair care composition which is capable of styling, straightening or conditioning hair by providing “virgin feel condition” to the hair after multiple washes and particularly provides at least one of the following results including (1) increased hydrophobicity, (2) improved detangling and wet combability, (3) improved deposition, and (4) no build-up.

In another non-limiting embodiment, the present application discloses the synergy of hydroxy ketone in hair straightening, hair styling or hair conditioning that lasts for longer duration. The synergistic combination of (i) at least one hydroxy ketone; (ii) at least one organic or inorganic base component facilitates the penetration of the composition into the hair fiber's cortex and modify the hair fiber's natural shape. Styling polymer, thickening polymer, conditioning polymer or biopolymer will react with itself and/or with the straightening components, i.e., hydroxy ketone or dihydroxy ketone to get semi-permanently affixed to hair and survive multiple shampoo cycles. In doing so, the hair tress will have significant improvement in providing or enhancing life to it and still maintain same desired aesthetic attributes like shine, conditioning, luster, and feel etc. After thermal activation and styling, these polymers are semi-permanently affixed to the hair and these effects will last a minimum of 10 shampoo and conditioning cycles when using the harsh sulfate based shampoos. Even longer lasting results are obtained when non-sulfate based shampoos are used as known to those skilled in the art, generally around 30 shampoo cycles. Furthermore, thermal activation of the composition does not produce formaldehyde or noxious vapors to achieve these effects and thereby provides a safer alternative to the formaldehyde based “Brazilian Blowout” treatment that is currently being banned across the globe.

Without being bound by the theory, it is further contemplated that the functional polymer further aids in permanent fixation onto the hair fiber. These polymers include acidic, alcoholic, thiol, or amino/amide bearing at least one hydrogen atom. The addition of diols and polyols will help increase hydroxyacetone penetration into hair and then create oligomers of polyketals with heat, which inhibit the penetration of water from outside to inside the hair. These molecules also react with the side chain of protein's amino-acids like lysine and arginine to create straighter hair (also crosslinks between keratin bundles). In addition, a three-fold increase in the rate of hair straightening was observed by diols, such as it went from 1 in/second to 3 in/second a flat iron rate. The presence of one or more of these groups on the straightening product will react with the straightening agents described to form a crosslinked matrix that remains affixed to hair for several shampoo cycles. The presence of the polymer and/or biopolymer on the hair after several shampoo cycles is quite evident by the feel of the treated fiber as compared to the control fiber containing all the same components, but lacks the polymer component in this composition.

In another non-limiting embodiment, the present application provides a hair straightening or hair styling composition comprising: (i) a hydroxy ketone selected from α-hydroxy ketone, β-hydroxy ketone, dihydroxy ketone, 4-hydroxy butanone, 1-hydroxy-2-butanone and combinations thereof; (ii) an organic or inorganic base selected from sodium bicarbonate, ammonium bicarbonate, guanidine carbonate and propylene carbonate; (iii) at least one functional polymer selected from PVM/MA Decadiene Crosspolymer, Amodimethicone, VA/Butyl Maleate/Isobornyl Acrylate Copolymer, Glycerin (and) Glyceryl Acrylate/Acrylic Acid Copolymer, and Guar hydroxypropyltrimonium chloride; (iv) at least one diol, polyol or sugar selected from propylene glycol, butanediol, dimethicone polyol, or combinations thereof; (v) at least one reducing agent sodium sulfite or ammonium sulfite; and (vi) at least one carrier, wherein said composition has a pH of about 5 to about 11.

In yet another non-limiting embodiment, the present application provides a hair straightening or hair styling composition comprising: (i) about 0.1 to about 70 wt. % of hydroxyacetone or dihydroxyacetone or combinations thereof; (ii) about 0.1 to about 20 wt. % of sodium bicarbonate and/or propylene carbonate; (iii) optionally, about 0.1 to about 20 wt. % of at least one functional polymer, and (iv) optionally, about 0.1 to about 20 wt. % of at least one diol, polyol, sugar, or combinations thereof; (v) optionally, about 0.1 to about 20 wt. % of at least one reducing agent and (vi) about 40 to about 95 wt. % of at least one carrier, wherein said composition has a pH of about 5 to about 11.

It is well known in the prior art that utilization of basic neutralizers like NaOH, or sodium phosphates, the straightening effect is not that pronounced as desired, and whereas, the present application discloses combining hydroxy acetone and sodium bicarbonate having long lasting straightening effect. Moreover, consumer safety is of premier importance, the neutral to slightly alkaline pH region for hair straightening is observed to be superior as compared to that of highly acidic or highly alkaline condition based straightening effects. The present hair styling composition displays method which can be applied on damp or dry hair, longer lasting straightening effects, shorter processing times as hair can be washed right away immediately once the hair is straightened unlike the formaldehyde treatment which requires long “waiting period” to wash the hair, straightening does not damage the hair, and is safer to scalp and hair.

The present composition enables treatment of hair at wide pH ranges of about 1-14. In another embodiment, the present composition enables treatment of hair in neutral to slightly alkaline conditions at pH 7-12 which is much safer and less corrosive to scalp and hair. The hair composition of the present application can be employed for oxidatively dyed hair without extreme color changes that are observed by the other hair straightening treatments even before the thermal activation step is applied. In this regard, lower straightening temperatures with multiple passes are required to minimally affect the dye molecules and to provide some straightening effects.

In yet another non-limiting embodiment, within scope and contemplation, the present hair care composition comprising at least one hydroxy ketone, at least one organic or inorganic base, and optionally, at least one functional polymer can be employed in combination with hair care compositions and components that are commercially available and very well known in relevant literature as prior art for a person skilled in the relevant art, and wherein, such non-limiting compositions that are known in the prior art include glyoxylic acid/dimethyl urea, polyacrylates cross polymer, polysaccharides, film forming agents and N-alkyl-2-mercaptoacetamide.

In yet another non-limiting embodiment, the present application discloses a method of straightening or styling hair, the method comprises: (a) washing hair tresses with shampoo; (b) eliminating or reducing the moisture of hair tresses of step (a); (c) applying hair straightening or styling composition comprising (i) about 0.1 to about 70 wt. % of at least one α or β-hydroxy ketone, or combinations thereof; (ii) about 0.1 to about 20 wt. % of at least one organic or inorganic base; (iii) optionally , about 0.1 to about 20 wt. % of at least one functional polymer; (iv) optionally, about 0.1 to about 20 wt. % of at least one diol, polyol, sugar, or combinations thereof; (v) optionally, about 0.1 to about 20 wt. % of at least one reducing agent; and (vi) about 40 to about 95 wt. % of at least one carrier, wherein said composition has a pH of about 5 to about 11, to the dried or damp hair of step (b); (d) leaving the treated hair for about 15 to 30 minutes for penetration; (e) hot air drying the treated hair of step (d) by mechanically straightening hair with a slow uniform motion from root to tip; (f) ironing the hair resulting from step (e) for at least 4 to 8 times using flat iron pre-heated to 375-450° F.; and (g) allowing the hair tresses as of step (f) to remain as such for at least 15 to 20 minutes to obtain straightened or styled hair.

According to one embodiment of the present application, the hair tresses are selected from Caucasian, Asian and African origins selected from fine, medium and coarse virgin, damaged, colored, grey, bleached hairs, and wherein, the hair tress has textures comprising wavy, curly, coily or kinky patterns.

According to one important embodiment of the present application, the composition employs molecules which are much safer and allows for the hair treatment for straightening, conditioning or styling to be performed at wide pH range of highly basic to highly acidic, and does not emit formaldehyde during the process, and is capable of providing long term hair straightening.

Advantageously, the hair straightening process of the present application provides a hair styling and/or hair straightening that lasts for at least 10 to 30 hair washes even after using the strong sulfate based shampoo and conditioner. Further, the combination of hydroxy ketone/s and organic/inorganic base penetrates into the hair fiber's cortex and modifies the hair fiber's natural shape. The functional polymer of the present application provides additional shape control, styling, shine, feel, conditioning, luster to the hair and etc.

Further, certain aspects of the present application are illustrated in detail by way of the following examples. The examples are given herein for illustration of certain aspects of the present application and are not intended to be limiting thereof.

### EXAMPLES

### Example 1

### Hair Straightening or Styling Composition 1

| Ingredients | Wt. % |
|---|---|
| Hydroxyacetone | 15.0 |
| Ammonium bicarbonate | 1.0 |
| Sodium Sulfite | 2.0 |
| Guar hydroxylpropyl | 0.4 |
| trimonium chloride | |
| Propylene glycol | 3.0 |
| Phenoxyethanol | 1.0 |
| Water | 76.6 |

Example 1 describes styling or straightening composition comprising hydroxyacetone, ammonium bicarbonate, cationic polymer guar hydroxylpropyl trimonium chloride, propylene glycol, phenoxyethanol, along with desired quantity of water.

### Example 2

### Hair Straightening or Styling Composition 2

| Ingredients | Wt. % |
|---|---|
| Hydroxyacetone | 8.0 |
| 1,3 propanediol | 2.0 |
| Sodium bicarbonate | 1.0 |
| Sodium bisulfite | 1.5 |
| Acrylate copolymer | 1.0 |
| Phenoxyethanol (and) Caprylyl glycol | 1.5 |
| Glycerin (and) Glyceryl acrylate/ | 1.5 |
| acrylic acid copolymer | |
| Water | 83.5 |

Example 2 describes styling or straightening composition comprising hydroxy acetone, 1,3-propanediol, sodium bicarbonate, sodium bisulfate, acrylate copolymer, phenoxyethanol and caprylyl glycol, glycerin and glyceryl acrylate, acrylic acid copolymer along with desired quantity of water.

### Example 3

### Hair Straightening or Styling Composition 3

| Ingredients | Wt. % |
|---|---|
| 4-hydroxy-2-butanone | 20.0 |
| Isopropyl alcohol | 4.0 |
| Sodium metabisulfite | 3.0 |
| Ammonium bicarbonate | 2.5 |
| Keratin (amino acid) | 1.5 |
| Phenoxyethanol (and) caprylyl glycol | 1.5 |
| VA/Butyl maleate/Isobornyl acrylate | 0.5 |
| copolymer | |
| Carbomer | 0.4 |
| Water | 66.6 |

Example 3 describes styling or straightening composition comprising 4-hydroxy butanone, isopropyl alcohol, sodium metabisulfite, ammonium bicarbonate, keratin, phenoxyethanol (and) caprylyl glycol, vinyl acetate/butyl maleate/isobornyl acrylate copolymer, Carbomer along with desired quantity of water.

### Example 4

### Hair Straightening or Styling Composition 4

| Ingredients | Wt. % |
|---|---|
| Hydroxyacetone | 10.0 |
| 1,3-propanediol | 10.0 |
| Sodium Bicarbonate | 1.0 |
| Sodium sulfite | 2.0 |
| Hydroxyethylcellulose | 1.0 |
| Amodimethicone | 2.0 |
| Phenoxyethanol | 1.0 |
| Water | 73.0 |

Example 4 describes styling or straightening composition comprising hydroxyacetone, 1,3-propanediol, sodium bicarbonate, sodium sulfite, hydroxyethylcellulose, amodimethicone, phenoxyethanol, along with desired quantity of water.

### Example 5

### Hair Straightening or Styling Composition 5

| Ingredients | Wt. % |
|---|---|
| 1-hydroxy-2-butanone | 5.0 |
| Hydroxyacetone | 8.0 |
| 1,3-propanediol | 5.0 |
| Propylene carbonate | 2.0 |
| Cysteine | 2.0 |
| Lysine | 1.0 |
| Paraben, Phenoxyethanol (and) | 1.0 |
| Caprylyl glycol | |
| PVM/MA Decadiene | 0.5 |
| Crosspolymer | |
| Water | 75.5 |

Example 5 describes styling or straightening composition comprising combination of 1-hydroxy-2-butanone and hydroxy acetone, base propylene carbonate; amino acids cysteine and lysine, preservative paraben, phenoxyethanol and caprylyl glycol mixture, polymer PVM/MA decadiene crosspolymer along with desired quantity of water.

### Example 6

### Hair Straightening or Styling Composition 6

| Ingredients | Wt. % |
|---|---|
| Hydroxyacetone | 15.0 |
| Dihydroxyacetone | 2.0 |
| Sodium thiosulfate | 1.0 |
| Guanadine carbonate | 1.0 |
| Butanediol | 1.0 |
| Dimethicone copolyol | 2.0 |
| Sodium polyacrylate | 1.5 |
| Phenoxyethanol, Caprylyl glycol | 1.0 |
| (and) Sorbic acid | |
| Water | 75.5 |

Example 6 describes styling or straightening composition comprising combination of hydroxy acetone, dihydroxyacetone, base sodium thio sulfate, guanidine carbonate, dimethicone copolyol, sodium polyacrylate, preservative phenoxyethanol, caprylyl glycol and Sorbic acid along with desired quantity of water.

### Example 7

### A Hair Straightening or Styling Method

Hair tress was washed thoroughly with shampoo and warm water (35-40° C.) followed by second wash, and rinsed for 30 seconds. Excess water was removed, and moisture was removed using blot with towel. Hair styling product of any of the above formulations of Examples 1 to 6 was applied; through from root to tip with fingers and comb until uniformly distributed to remove excess. Treated hair was left for 30 minutes for the styling product to penetrate. (Hang tress in straight position). After 30 minutes, tress was hot blow-dried as straight as possible with much tension using a round brush or comb with slow uniform motion from root to tip. Flat iron was pre-heated and hair was treated with pre-heated flat iron from root to tip about six times. Allowed tress to equilibrate to lab conditions; Evaluated tress state: Straightened. Washed hair tress with warm water (35-40° C.) for 30 second wetting, 30 seconds wash (shampoo), and 30 seconds rinse. Hair was conditioned for 30 seconds, followed by 30 seconds rinse. After hair wash, hair was dried to see hair straightened.

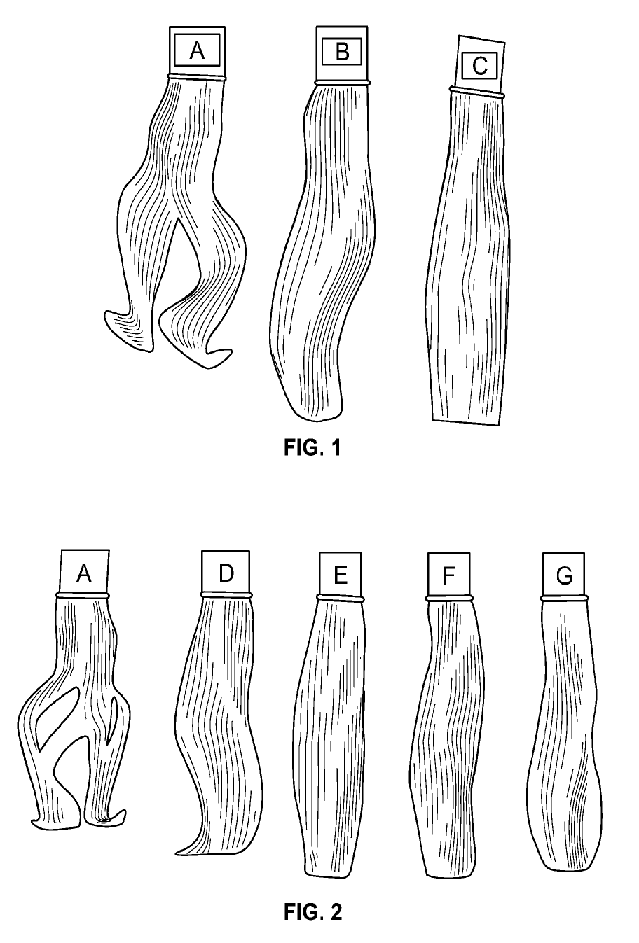

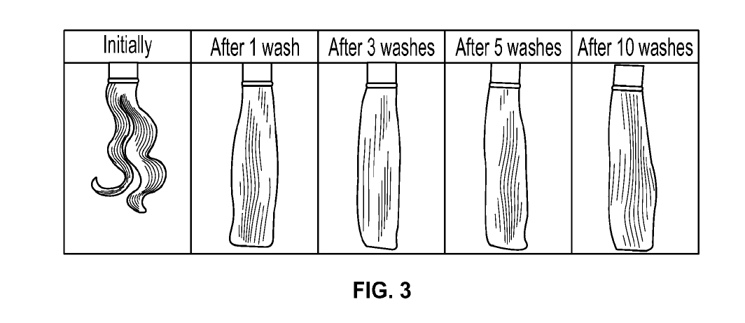

The results of treating natural hair are shown in FIGS. 1, 2 and 3. It is observed that the styled or straightened hair remains in the same state even after 10 wash cycles. FIG. 1 shows the results of treating untreated hair (A) with dihydroxy acetone at pH 3.9 (B) and with dihydroxy acetone and sodium bicarbonate at pH 7.5 (C). FIG. 2 shows the results of treating hair with hydroxy acetone at pH 3.5 (D); hydroxy acetone and sodium bicarbonate at pH 8.1 (E); hydroxy acetone and sodium tetra borate dehydrate at pH 8.3 (F); and hydroxyacetone and dibasic potassium phosphate at pH 8.2 (G). FIG. 3 shows the results of treating hair hydroxy acetone and sodium bicarbonate at pH 8.1 for every 0, 1, 3, 5 and 10 wash cycles. It has been observed that the hair remains in the straightened or styled state even after 10 wash cycles.

While this invention has been described in detail with reference to certain preferred embodiments, it should be appreciated that the present invention is not limited to those precise embodiments. Rather, in view of the present disclosure, which describes the current best mode for practicing the invention, many modifications and variations would present themselves to those skilled in the art without departing from the scope and spirit of this invention.

What is claimed is:

1. A hair straightening or hair styling composition comprising:
i. about 0.1 to about 70 wt. % of at least one hydroxy ketone, dihydroxy ketone or combinations thereof;
ii. about 0.1 to about 20 wt. % of at least one organic or inorganic base;
iii. optionally about 0.1 to about 20 wt. % of at least one functional polymer;
iv. optionally about 0.1 to about 20 wt. % of optionally at least one diol, polyol, sugar, or combinations thereof;
v. optionally about 0.1 to about 20 wt. % of at least one reducing agent; and
vi. about 40 to about 95 wt. % of at least one carrier; wherein said composition has a pH of about 5 to about 11.

2. The hair straightening or hair styling composition according to claim 1, wherein said functional polymer is used in (i) styling and thickening, (ii) thickening and conditioning, (iii) styling and conditioning or (iv) styling, thickening and conditioning.

3. The hair straightening or hair styling composition according to claim 1, wherein said composition further comprises about 0.1 to about 20 wt. % of at least one protein or amino acid component selected from the group consisting of di-peptide to decapeptide, diols, polyols, sugars, alpha and/or beta-keratins, silk proteins, wheat protein, oat protein, milk protein, glycoprotein, soy protein, barley protein, quinoa protein, collagen protein, vegetable protein, chickpea protein, avacado protein, egg protein, honey protein, rice protein, casein, light protein, gelatin, and their hydrolyzed counterparts.

4. The hair straightening or hair styling composition according to claim 1, wherein said composition further comprises at least one cosmetically acceptable ingredient selected from the group consisting of silicones, reducing agents, oxidizing agents, conditioning agents, humectants, pH adjusting buffers, petrolatum thickeners, mineral oils, emulsifiers, fragrances, and preservatives.

5. The hair straightening or hair styling composition according to claim 1, wherein said hydroxy ketone is selected from the group consisting of α-hydroxy ketone, β-hydroxy ketone, 4-hydroxy-2-butanone, 1-hydroxy-2-butanone and combinations thereof.

6. The hair straightening or hair styling composition according to claim 1, wherein said organic or inorganic base is an organic or inorganic carbonates or bicarbonates selected from the group consisting of sodium bicarbonate, potassium bicarbonate, disodium or dipotassium carbonate, sodium potassium carbonate; lithium, magnesium, calcium, zinc, aluminum, guanidine, or imidazolium carbonates or bicarbonates; diammonium carbonate, ammonium bicarbonate or ammonium sodium carbonate; alkyl or aryl ammonium bicarbonates;
propylene carbonate, dialkyl ammonium carbonates or bicarbonates; alkyl aryl ammonium carbonates or bicarbonates; diaryl ammonium bicarbonates, trialkyl ammonium carbonates or bicarbonates; alkyl aryl ammonium carbonate or triaryl ammonium bicarbonates, hydroxyl alkyl or hydroxyl aryl ammonium bicarbonates; dihydroxyl alkyl ammonium carbonates or bicarbonates; hydroxyl alkyl or hydroxyl aryl ammonium carbonates and bicarbonates; dihydroxyl hydroxyl aryl ammonium bicarbonates, trihydroxyl alkyl ammonium carbonates or bicarbonates; hydroxyl alkyl hydroxyl aryl ammonium carbonate; trihydroxyl aryl ammonium bicarbonates, and wherein the alkyl or hydroxyl alkyl ammonium groups are linear or branched from carbon length C₁-C₃₀, hydrophobic chain lauryl, stearyl, behenyl and alkylaryl; or is organic or inorganic alkali hydroxides, sulfates, borates, manganates, aluminates, silicates, phosphates, alone or in combinations thereof.

7. The hair straightening or hair styling composition according to claim 2, wherein said functional polymer is a polymer or blend of polymers from synthetic and/or natural origin with weight-average molecular mass from about 500 to about 20,000,000 daltons containing at least one or more of the following monomeric components selected from the group consisting of styrenics, and/or sulfonated styrenics, acrylates, methacrylates, acrylamides, methacrylamides, vinyl lactams, vinyl pyrrolidone, vinyl caprolactam, vinyl amides, urethanes, ethoxylates, propoxylates, vinyl carboxylates, vinyl esters, vinyl ethers, maleates, itaconates, crotonates, and olefinics.

8. The hair straightening or hair styling composition according to claim 1, wherein said functional polymer is a polymer or blend of polymers derived from synthetic and/or natural origin with weight-average molecular mass from about 500 to about 20,000,000 daltons containing at least one or more of the following monomeric components selected from the group consisting of styrenics, and/or sulfonated styrenics, acrylates, methacrylates, acrylamides, methacrylamides, vinyl lactams, vinyl pyrrolidone, vinyl caprolactam, vinyl amides, urethanes, (meth)acroyl ethoxylates, (meth)acroyl propoxylates, vinyl carboxylates, vinyl esters, vinyl ketones, vinyl carbonates, vinyl acetals (meth)acroyl alkyl sulfonates, vinyl alkyl sulfonates, vinyl alkyl phosphates, (meth)acroyl alkyl phosphates, vinyl ethers, maleates, itaconates, crotonates, and olefinics.

9. The hair straightening or hair styling composition according to claim 8, wherein said functional polymer is a polysaccharides containing α-1,4 and/or β-1,4-glycosidic linkages with optionally α-1,6 and/or β-1,6 linkages, or optionally modified with (i) hydroxyalkyl, (ii) cationic functionality, (iii) hydrophobic groups including methyl, ethyl, propyl, and/or carboxymethyl groups, or (iv) hydrophilic groups including hydroxy ethyl, hydroxyl propyl, acetates, succinates, phosphates, sulfates and/or carbonates.

10. The hair straightening or hair styling composition according to claim 8, wherein said functional polymer is selected from the group consisting of cellulose, starch, guar, alginate, carrageen gum, cassia gum, and tarragum.

11. The hair straightening or hair styling composition according to claim 1, wherein said diol is a C₃-C₁₂ diol selected from the group consisting of propanediol, butane diol, pentane diol, and hexane diol; said polyol is a C₃-C₁₂ polyol selected from the group consisting of glycerol, erythritol, threritol pentaerythritol, mannitol, maltitol, glucitolxylitol, sorbitol, diethylene glycol, triethylene glycol, and dipropylene glycol; and said sugar is a C₃-C₁₂ sugar selected from the group consisting of glyceraldehyde, erythrose, threose, ribose, arabinose, xylose, lyxose, allolose, altrose, glucose, mannose, glucose, idose, galactose, talose, fructose, maltose, lactose; and combinations thereof.

12. The hair straightening or hair styling composition according to claim 1, wherein said reducing agent is selected from the group consisting of alkali metal sulfite, meta-bisulfite, ammonium sulfite, an alkali metal dithionite, cysteine, cystamine, thiols, thioethanol, dithioethane and thioglycolate.

13. The hair straightening or hair styling composition according to claim 1, wherein said carrier is water, alcohol, hydro alcoholic, an emulsion, an ester, a hydrocarbon or an organic or inorganic oil carrier.

14. The hair straightening or hair styling composition according to claim 1, wherein said composition optionally comprises a cross-linking monomeric unit having alcohol, thiol, carboxylic acid or anhydride, amino or ammonium, modified silicone or siloxane, alkoxysilane or, sulfate group functionality.

15. The hair straightening or hair styling composition according to claim 1, wherein said composition is regulatory compliant, safe, does not contain formaldehyde and acts on both cuticle and cortex of hair to make hair straightening or styling that lasts for at least 10 to 30 wash cycles.

16. The hair straightening or styling composition according to claim 1 formulated as an emulsion, lotion, gel, vesicle dispersion, paste, cream, solid stick, mousse, shampoo, spray, balm, wipe, milk, foam, jellies, liquid, tonics, and/or enamel.

17. A hair straightening or hair styling composition comprising:
i. about 0.1 to about 70 wt. % of α-hydroxyacetone, β-hydroxyacetone, dihydroxy acetone, 4-hydroxy butanone, 1-hydroxy-2-butanone and combinations thereof;
ii. about 0.1 to about 20 wt. % sodium bicarbonate;
iii. optionally about 0.1 to about 20 wt. % of at least one functional polymer;
iv. optionally about 0.1 to about 20 wt. % of at least one diol, polyol, sugar, or combinations thereof;
v. optionally about 0.1 to about 20 wt. % of at least one reducing agent; and
vi. about 40 to about 95 wt. % of at least one carrier;
wherein said composition has a pH of about 5 to about 11.

18. A method of straightening or styling hair, the method comprising:
(a) washing hair tresses with shampoo;
(b) eliminating or reducing the moisture of hair tresses of step (a);
(c) applying a hair straightening or styling composition comprising: (i) about 0.1 to about 70 wt. % of at least one α-hydroxyacetone, β-hydroxyacetone, dihydroxy acetone, 4-hydroxy butanone, 1-hydroxy-2-butanone and combinations thereof; (ii) about 0.1 to about 20 wt. % of at least one organic or inorganic base; (iii) optionally about 0.1 to about 20 wt. % of at least one functional polymer; (iv) optionally about 0.1 to about 20 wt. % of at least one diol, polyol, sugar, or combinations thereof; (v) optionally about 0.1 to about 20 wt. % of at least one reducing agent; and (vi) about 40 to about 95 wt. % of at least one carrier, wherein said composition has a pH of about 5 to about 11, to the dried or damp hair of step (b);
(d) leaving the treated hair for about 15 to 30 minutes for penetration;
(e) hot air drying the treated hair of step (d) by mechanically straightening with a slow uniform motion from root to tip;
(f) ironing the hair resultant of step (e) for at least 4-8 times using flat iron pre-heated to 375-450° F.; and
(g) allowing the hair tresses as of step (f) to remain as such for at least 15 to 20 minutes to obtain straightened or styled hair.

19. The method according to claim 18, wherein said hair tresses are selected from Caucasian, European, Asian and African origins having fine, medium and coarse virgin, damaged, colored, grey or bleached hairs and textures comprising wavy, curly, coily or kinky patterns.

20. The method according to claim 18, wherein,
(i) the method provides a 3-fold increase in rate of hair straightening;
(ii) the method allows straightened hair to be washed immediately;
(iii) the method acts on both dry and damp hair; and/or
(iv) the resultant straightened or styled hair remains in same state or condition for at least 15 to 30 hair washes even after using the sulfate based shampoo, and/or conditioner.
